(19) United States
(12) Patent Application Publication
(10) Pub. No.: US 2023/0033849 A1
(43) Pub. Date: Feb. 2, 2023

(54) CBD SUNSCREEN FORMULATIONS AND USES THEREOF

(71) Applicant: JUPITER WELLNESS, INC., Jupiter, FL (US)

(72) Inventors: Glynn WILSON, Jupiter, FL (US); Brian JOHN, Jupiter, FL (US); Richard MILLER, Jupiter, FL (US)

(21) Appl. No.: 17/938,613

(22) Filed: Oct. 6, 2022

Related U.S. Application Data
(63) Continuation of application No. PCT/US2021/025947, filed on Apr. 6, 2021.

(51) Int. Cl. A61K 8/34; A61K 8/49; A61K 8/42; A61K 8/35; A61K 8/368; A61K 8/40; A61K 8/27; A61K 8/29; A61Q 17/04

(52) U.S. Cl. CPC A61K 8/347; A61K 8/498; A61K 8/42; A61K 8/35; A61K 8/368; A61K 8/40; A61K 8/27; A61K 8/29; A61Q 17/04; A61K 2800/87

(57) ABSTRACT

Provided herein are topical formulations including a cannabinoid and one or more active sunscreen ingredients. Also provided are methods of using the topical formulations.

### BACKGROUND

Skin cancer is the most common of all human cancer. Skin cancers include melanoma skin cancer and non-melanoma skin cancers such as basal cell carcinoma and squamous cell carcinoma. Skin cancer prevention methods include sunlight avoidance and application of sunscreen. However, many active sunscreen ingredients may be harmful to human health and harmful to the environment. New sunscreens that are safer for humans and the environment are needed.

### BRIEF SUMMARY

The present disclosure provides a topical formulation comprising a cannabinoid and one or more active sunscreen ingredients that do not include oxybenzone. The cannabinoid may be a phytocannabinoid, an endocannabinoid, or a non-naturally occurring cannabinoid.

The cannabinoid may be a phytocannabinoid, such as Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabiolsoin (CBE), cannabicitran (CBT), or combination thereof. In particular embodiments, the phytocannabinoid includes CBD.

The cannabinoid may be an endocannabinoid, such as anandamide.

The cannabinoid may be a non-naturally occurring cannabinoid, such as CP55,940, WIN 55, 212-2, or nabilone.

The cannabinoid may be present in the topical formulation at a concentration of about 1 mg/ml to about 10 mg/ml. The cannabinoid may be included in the topical formulation in the form of a cannabinoid isolate having a total cannabinoid content of at least 95% (w/v). Additionally, the cannabinoid may have a THC content of less than 0.3%.

The one or more active sunscreen ingredients included in the topical formulation may include at least one chemical sunscreen or at least one mineral sunscreen.

The at least one chemical sunscreen may include avobenzone, homosalate, octisalate, octocrylene, or a combination thereof. The one or more active sunscreen ingredients may include avobenzone at a concentration of about 0.5% to about 10% weight by volume (w/v). The one or more active sunscreen ingredients may include homosalate at a concentration of about 5% to about 25% weight by volume (w/v). The one or more active sunscreen ingredients may include octisalate at a concentration of about 0.5% to about 10% weight by volume (w/v). The one or more active sunscreen ingredients may include octocrylene at a concentration of about 2% to about 20% weight by volume (w/v).

The at least one mineral sunscreen may include one or both of zinc oxide or titanium dioxide. The one or more active sunscreen ingredients may include zinc oxide at a concentration of about 5% to about 15% weight by volume (w/v). The one or more active sunscreen ingredients may include titanium dioxide at a concentration of about 5% to about 15% weight by volume (w/v).

The one or more active sunscreen ingredients may comprise, consist essentially of, or consist of homosalate, octisalate, octocrylene, and zinc oxide.

The one or more active sunscreen ingredients may comprise, consist essentially of, or consist of avobenzone, homosalate, octisalate, and octocrylene. The sun protection factor (SPF) of the topical formulation may be from about 5 to about 100, or from about 10 to about 55.

In certain embodiments, the topical formulations provided herein do not contain abobenzone.

The topical formulation may further include N-L-alpha-aspartyl-L-phenylalanine 1-methyl ester (APM) or the lower alkyl derivative thereof. The concentration of APM or its lower alkyl derivative in the topical formulation may be at a concentration of about 0.05% to about 5% (w/v), preferably about 0.2 to about 2% (w/v). The ratio of APM or its lower alkyl derivative to the cannabinoid in the topical formulation may be in the range of about 4:1 to about 10:1 (by weight).

The topical formulation may further include an emulsifier, a waterproofing agent, an emollient, a solvent, a perfume, an antioxidant, a preservative, or a combination thereof.

In some embodiments, the topical formulation has a pH of about 5 to about 7.

The topical formulation may be formulated in the form of a balm, a lotion, a liquid, or a gel.

In some aspects, the present disclosure provides a packaged formulation of a topical formulation described herein, contained in a hemp-based polymer package. The hemp-based polymer may include hemp propylene, hemp ethylene, hemp butylated hydroxytoluene, hemp acrylonitrile butadiene styrene, or a combination thereof.

In some aspects, the present disclosure provides a method of protecting the skin of a subject from sunburn by administering to the subject an effective amount of a topical formulation as described herein.

In some aspects, the present disclosure provides a method of protecting skin of a subject from ultraviolet (UV) radiation, comprising administering to the subject an effective amount of a topical formulation as described herein.

In some embodiments, administering the topical formulation is performed prior to exposure of the skin to UV radiation.

Administering the topical formulation may result in a reduced likelihood of the skin undergoing carcinogenesis. Administering the topical formulation may result in a reduced likelihood of a melanocyte or a keratinocyte in the skin undergoing carcinogenesis.

In some embodiments, the methods described herein protect the skin from one or both of UVa radiation and UVb radiation.

Other objectives, advantages and novel features of the disclosure will become more apparent from the following detailed description.

### BRIEF DESCRIPTION OF THE DRAWINGS

FIG. 1 shows MTT cell viability assay for UVB dose determination at increasing exposure times. Keratinocyte percent cell viability is graphed on Y axis and UVB irradiation exposure times is expressed in seconds on X axis.

FIG. 2 shows the effects of CBD on keratinocyte cell viability following UVB exposure, as determined by MTT assay. Cell viability was compared between cells without CBD treatment and cells treated with 3 different concentrations of CBD isolate. All the samples were irradiated with UVB lamp delivering 60 mJ/cm² radiation. Maximum absorbance for the MTT assay is 1.2, and the data were normalized to max absorbance.

FIG. 3 shows the effects of CBD on melanocytes cell viability following UVB exposure, as determined by MTT assay. Cell viability was compared between cells without CBD treatment and cells treated with 3 different concentrations of CBD isolate. All the samples were irradiated with UVB lamp delivering 60 mJ/cm² radiation. Maximum absorbance for MTT assay is 1.2, and the data were normalized to max absorbance.

### DETAILED DESCRIPTION

Presented herein are topical formulations comprising and one or more active sunscreen ingredients that do not include oxybenzone. In certain embodiments, the topical formulations are used in methods of protecting skin from sunburn. Additionally, the combination of the cannabinoid and the one or more active sunscreen ingredients in the topical formulations provides a non-toxic method of reducing risk of cancer caused by exposure to ultraviolet radiation.

As used herein, a “sunscreen” is a dermatological formulation that coats skin, thereby preventing certain UV radiation from causing the skin to sunburn.

As used herein, “sunburn” refers to a type of skin burn resulting from too much exposure to sunlight or sunlamps. Symptoms may include red, painful, itchy skin, skin that feels hot to the touch, and blistering. Skin reddening is the most characteristic symptom of sunburn.

In the present description, the term “about” means±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

### Cannabinoids

Provided herein are topical formulations and uses of topical formulations that include a cannabinoid and one or more active sunscreen ingredients that do not include oxybenzone.

The term “cannabinoid” refers to a class of compounds that bind to one or more cannabinoid receptors and act on the endocannabinoid system. Cannabinoids include phytocannabinoids, endocannabinoids, and non-naturally occurring cannabinoids. The endocannabinoid system is a biological system present in mammals that includes endocannabinoids, which are lipid based neurotransmitters that bind to cannabinoid receptors. Cannabinoid receptor 1 (CB1) and cannabinoid receptor 2 (CB2) are expressed in the central and peripheral nervous system, and cannabinoid receptor 3 (CB3) is expressed is the central nervous system. Other non-classical cannabinoid receptors include G protein-coupled receptor (GPR55), GRP119 and GPR18, peroxisome proliferator-activated receptors (PPARs) and transient receptor potential vanilloid 1 (TRPV1).

Endocannabinoid signaling through cannabinoid receptors affect cognitive processes such as mood, appetite, and memory. Cannabinoids are also present on a variety of other cells types and tissues. For example, CB2 is expressed on monocytes, macrophages, and B and T cells.

In certain embodiments, the cannabinoid is a phytocannabinoid. A phytocannabinoid is a cannabinoid that is naturally produced by a plant. Phytocannabinoids are typically C21 or C22 (for the carboxylated forms) terpenophenolic compounds. Plants that produce cannabinoids include Cannabis, Echinacea purpurea, Echinacea angustifolia, Acmelia oleracea, Helichrysum umbracaligerum, and Radula marginata. Examples of phytocannabinoids include dodeca-2E, 4E, 8Z, 10E/Z-tetraneoic-acid-isobutylamid, beta-caryophyllene, perottetinene, Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT).

In certain embodiments, the phytocannabinoid comprises a Cannabis-derived phytocannabinoid. Cannabis generally refers to the plant genus that includes Cannabis saliva, Cannabis saliva forma indica, and Cannabis ruderalis. Examples of phytocannabinoids produced by Cannabis include Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabielsoin (CBE), and cannabicitran (CBT) (see, e.g., Prandi et al., Molecules 23(7), 1526, 2018). Cannabis-derived cannabinoids accumulate in secretory cavities of trichomes, which are present in the female flower of the plant. Cannabinoids may also be present in lower concentrations in seeds, roots, and stems of the plant. Many cannabis strains have either THCA or CBDA as the predominant cannabinoid produced, although it is typical for a variety of cannabinoids to be present together. When THCA and CBDA are decarboxylated, such as through heat treatment, the molecules are converted to THC and CBD, respectively.

In certain embodiments, the cannabis-derived phytocannabinoid comprises CBD. In some embodiments, the cannabis-derived phytocannabinoid comprises CBD and at least one other cannabis-derived phytocannabinoid. As demonstrated herein, CBD provides a surprising protective effect against UV radiation for melanocytes and keratinocytes. These protective effects demonstrated indicate the usefulness of CBD in sunscreen formulations, particularly given that CBD is hydrophobic and therefore readily penetrates the skin.

In certain embodiments, the cannabinoid comprises an endocannabinoid. Endocannabinoids are lipid-based neurotransmitters that are endogenously expressed and bind to cannabinoid receptors of the endocannabinoid system. Examples of endocannabinoids include anandamide, arachidonoyl-ethanolamide (AEA), 2-arachidonoyl-glycerol (2-AG), 2-arachidonyl glyceryl ether (noladin ether), N-arachidonoyl domain (NADA), virodhamine (OAE), and lysophosphatidylinositol (LPI). In certain embodiments, the endocannabinoid comprises anandamide.

In certain specific embodiments, the cannabinoid comprises a non-naturally occurring cannabinoid (also referred to as “synthetic cannabinoid”). Examples of non-naturally occurring cannabinoids include CP55,940, which is a potent THC mimic; WIN 55, 212-2 (which is an aminoalkylindole derivative with cannabinoid receptor agonist activity), nabilone (which is structurally very similar to THC), JWH-018 (1-pentyl-3-(1-naphthoyl)indole), dimethylheptylpyran, HU-210 (which is about 100 times as potent as THC), HU-331 (which is a quinone anticancinogenic drug synthesized from cannobidiol), JWH-133 (which is a potent selective CB2 receptor agonist), Levonantradol (Nantrodolum), or AM-2201 (which is a potent cannabinoid receptor agonist). In certain particular embodiments, the synthetic cannabinoid comprises CP55,940. WIN 55, 212-2, or nabilone.

In some embodiments, the cannabinoid is provided in the formulation at a concentration in the range of about 0.05 mg/ml to about 100 mg/ml. In certain embodiments, the cannabinoid is provided in the formulation at concentration in the range of about 0.1 mg/ml to about 100 mg/ml. In particular embodiments, the cannabinoid is provided in the concentration at a range of between about 1 mg/ml and about 20 mg/ml, such as about 1 mg/ml to about 8 mg/ml, about 1 mg/ml to about 5 mg/ml, about 5 mg/ml to about 10 mg/ml, about 10 mg/ml to about 15 mg/ml, about 15 mg/ml to about 20 mg/ml, about 1 mg/ml to about 10 mg/ml, or about 10 mg/ml to about 20 mg/ml. In certain embodiments, the cannabinoid is provided in the formulations at about 100 μg/ml to about 200 μg/ml, about 200 μg/ml to about 300 μg/ml, about 300 μg/ml to about 400 μg/ml, about 400 μg/ml to about 500 μg/ml, about 500 μg/ml to about 600 μg/ml, about 600 μg/ml to about 700 μg/ml, about 700 μg/ml to about 800 μg/ml, about 800 μg/ml to about 900 μg/ml, about 100 μg/ml to about 500 μg/ml or about 500 μg/ml to about 1000 μg/ml. In certain embodiments, the cannabinoid is provided in the formulations at about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, or about 10 mg/ml. In certain embodiments, the cannabinoid is provided in the formulations at about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, or about 20 mg/ml.

### Active Sunscreen Ingredients

As previously noted, provided herein are topical formulations and uses of formulations that include a cannabinoid and one or more active sunscreen ingredients that do not include oxybenzone.

Oxybenzone is a commonly used active sunscreen ingredient that has been found in lab studies to have weak estrogen and anti-androgenic effects, and is absorbed into the bloodstream of humans following application. Additionally, oxybenzone has been found to have harmful effects on coral reefs.

In certain embodiments, the one or more active sunscreen ingredients include at least one chemical sunscreen agent. These chemical agents are generally organic compounds that absorb energy of UV radiation, causing the compound to become excited to a higher energy state. The organic compounds will then return to a lower energy ground state with concomitant loss of energy as heat.

Chemical suncreen ingredients may include p-aminobenzoic acid (PABA), padimate O (also known as OD-PABA), phenylbenzimidazole sulfonic acid, cinoxate, homosalate, menthyl anthranilate, octocrylene, octyl methoxycinnamate, octyl salicylate, sulisobenzone, trolamine salicylate, avobenzone, emcansule, 4-methylbenzylidene camphor, bisoctrizole (also known as methylene bis-benzotriazolyl tetramethylbutylphenol), bis-ethylhexyloxyphenol methoxyphenol triazine (also known as bemotrizinol), tris-biphenyl triazine, bisdisulizole disodium, drometrizole trisiloxane, sodium dihydroxy dimethoxy disulfobenzophenone, octyl triazone, diethylamino hydroxybenzoyl hexyl benzoate, iscotrizinol, dimethico-diethylbenzalmalonate, and isopentyl-4-methoxycinnamate. In some embodiments, the chemical sunscreen agent is selected from avobenzone, homosalate, octisalate, octocrylene, or a combination thereof. In certain other embodiments, the chemical sunscreen agent is selected from homosalate, octisalate, octocrylene, or a combination thereof.

In particular embodiments, the at least one chemical sunscreen agent comprises avobenzone. The avobenzone may be at a concentration of about 0.5% to about 10% weight by volume (w/v).

In certain other embodiments, the at least one chemical sunscreen agent does not comprise avobenzone.

In particular embodiments, the at least one chemical sunscreen agent comprises homosalate. The homosalate may be at a concentration of about 5% to about 25% weight by volume (w/v).

In particular embodiments, the at least one chemical sunscreen agent comprises octisalate. The octisalate may be at a concentration of about 0.5% to about 10% weight by volume (w/v).

In particular embodiments, the at least one chemical sunscreen agent comprises octocrylene. The octocrylene may be at a concentration of about 2% to about 20% weight by volume (w/v).

In certain embodiments, the one or more active sunscreen ingredients include at least one mineral sunscreen agent. Mineral sunscreen agents are generally UV blockers that coat the skin and reflect and scatter UV radiation, thus forming a barrier between the UV radiation and the skin. The at least one mineral sunscreen agent may be one or both of zinc oxide and titanium dioxide.

In particular embodiments, the at least one mineral sunscreen agent comprises zinc oxide. The zinc oxide may be present at a concentration of about 5% to about 15% weight by volume (w/v).

In particular embodiments, the at least one mineral sunscreen agent comprises titanium dioxide. The titanium dioxide may be present at a concentration of about 5% to about 15% weight by volume (w/v).

In certain embodiments, the one or more active sunscreen ingredients include only one or more chemical sunscreen agents. In certain other embodiments, the one or more active sunscreen ingredients include only one or more mineral sunscreen agents. In yet other embodiments, the one or more active sunscreen ingredients include at least one chemical sunscreen agent and at least one mineral sunscreen agent.

In some embodiments, the one or more active sunscreen ingredients comprise avobenzone, homosalate, octisalate, and octocrylene. In some embodiments, the one or more active sunscreen ingredients consist of or consist essentially of avobenzone, homosalate, octisalate, and octocrylene. In some embodiments, the one or more active sunscreen ingredients comprise homosalate, octisalate, octocrylene, and zinc oxide. In some embodiments, the one or more active sunscreen ingredients consist of or consist essentially of homosalate, octisalate, octocrylene, and zinc oxide.

In some embodiments, the one or more active sunscreen ingredients comprise an N-L-alpha-aspartyl-L-phenylalanine 1-methyl ester (APM) or a lower alkyl derivative thereof. N-L-alpha-aspartyl-L-phenylalanine 1-methyl ester (APM) may also be referred to as aspartame. The term “lower alkyl derivative of APM” refers to a compound where the methyl group of the 1-methyl ester of APM is replaced with an alkyl group having 2-4 carbons, such as ethyl, propyl, isopropyl, or butyl.

In some embodiments, the concentration of APM or a lower alkyl derivative thereof is about 0.05% to about 2% (w/v), such as about 0.2 to about 2% (w/v), about 0.2% to about 0.4%, about 0.4% to about 0.6%, about 0.6% to about 0.8%, about 0.8% to about 1.0%, about 1.0% to about 1.2%, about 1.2% to about 1.4%, about 1.4% to about 1.6%, about 1.6% to about 1.8%, or about 1.8% to about 2.0% (w/v). Preferably, the APM or lower alkyl derivative is at a concentration of about 0.5% to about 1.5% (w/v).

In some embodiments, the ratio of APM or a lower alkyl derivative thereof to the cannabinoid in the formulation is in the range of about 4:1 to about 10:1 (by weight). In some embodiments, the ratio of APM or a lower alkyl derivative thereof to the cannabinoid in the formulation is about 4:1 to about 5:1, about 5:1 to about 6:1, about 6:1 to about 7; 1, about 7:1 to about 8.1, about 8:1 to about 9:1, or about 9:1 to about 10:1 (by weight). In some embodiments, the ratio of APM or a lower alkyl derivative thereof to the cannabinoid in the formulation is in the range of about 5:1 to about 8:1 (by weight)

### Formulations

As previously noted, provided herein are topical formulations comprising a cannabinoid and at least one active sunscreen ingredient that does not include oxybenzone. Such formulations include dermatological formulations. Dermatological formulations may include a dermatologically acceptable carrier.

In certain embodiments, the cannabinoid is provided in the formulation in the form of a cannabinoid isolate. The term “cannabinoid isolate” refers to a highly purified cannabis-derived cannabinoid. A cannabinoid isolate may be produced, for example, by CO₂ extraction, ethanol extraction, or butane extraction. Physical forms of a cannabinoid isolate include, for example, a crystal, a powder, a wax, or a resin. A cannabinoid isolate may have a total cannabinoid content of at least 65%, at least 70%, at least 75%, at least 80%, at least 85%, at least 90%, or at least 95% cannabinoid (w/v). In certain embodiments, the cannabinoid isolate has a total cannabinoid content of at least 95% (w/v). In certain embodiments, the cannabis-derived phytocannabinoid comprises a cannabinoid isolate having a THC content of less than 0.3%.

As used herein, “carrier” and “physiologically acceptable carriers” are used interchangeably and include any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, stabilizers, polymers, gels, binders, dyes, such like materials and combinations thereof, as would be known to one of ordinary skill in the art and are molecular entities and compositions that are generally non-toxic to recipients at the dosages and concentrations employed, i.e., do not produce an adverse, allergic or other untoward reaction when administered to an animal, such as a human, as appropriate (see, for example, Remington's Pharmaceutical Sciences, 18th Ed. Mack Printing Company, 1990, pp. 1289-1329, incorporated herein by reference). Except insofar as any conventional carrier is incompatible with an active ingredient, its use in a topical formulations provided herein is contemplated. In certain embodiments, the carrier is suitable to be included in dermatological formulations.

Topical formulations may be in the form of, for example, solutions, suspensions, foam, lotions, gels, pastes, medicated sticks, balms (e.g., lip balm), spray, powders (e.g., body powder or baby powder), creams or ointments. Such formulations optionally contain humectants, emollients, absorption enhancing agents, solubilizers, stabilizers, tonicity enhancing agents, buffers, preservatives, and/or additional therapeutic agents.

In some embodiments, the topical formulation includes an emulsifier, a waterproofing agent, an emollient, a solvent, a perfume, an antioxidant, a preservative, or a combination thereof.

In embodiments, the topical formulations include a waterproofing agent. Waterproofing agents allow the topical formulation to remain adhered to skin when submerged in water and include waxes, oils, and certain polymers such as dimethicone.

In embodiments, the topical formulations include an emulsifier. The emulsifier may include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, oleamide DEA, sodium glyceryl oleate phosphate, hydrogenated vegetable glycerides phosphate PEG-20 stearate, PEG-30 dipolyhydroxystearate, PEG-40 hydrogenated castor oil, PEG-60 hydrogenated castor oil, PEG-40 stearate, PEG-100 stearate, polyglyceryl-3 methylglucose distearate, polysorbate 20, polysorbate 60, polysorbate 80, potassium cetyl phosphate, DEA cetyl phosphate, ethomeen, dimethicone PEG-7 phosphate, or a combination thereof.

In embodiments, the topical formulations include an emollient. A topical formulation may include an emollient to have a softening or soothing effect on the skin. In certain particular embodiments, the emollient may include Simmondsia chinensis seed oil, glycerine, sorbitol, propylene glycol, isopropyl myristate, isopropyl palmitate, butylene glycol, caprys caprylic triglyceride, coco-caprylate/caprate, cocoglycerides, mineral oil, lanolin oil, coconut oil, cocoa butter, olive oil, aloe extract, banana fruit extract, jojoba oil, castor oil, a fatty acid such as oleic or stearic, fatty alcohol such as cetyl and hexadecyl, behenyl alcohol, diisopropyl adipate, hydroxybenzoate ester, benzoic acid ester, a C₁₀-C₁₆ alcohol, isononyl iso-nonanoate, a silicone such as dimethyl polysiloxane, caprylyl methicone, an ether such as polyoxypropylene butyl ethers or polyoxypropylene cetyl ether, C₁₂-C₁₅ alkyl benzoates, 1,2-hexanediol, acetyl glucosamine, or any combination thereof. Examples of concentration ranges that the emollients may be provided in include about 0.1% to about 10%, or about 0.5% to about 5% (w/v).

In embodiments, the topical formulation may include a preservative. For example, preservatives can be present in a gelled formulation to minimize bacterial and/or fungal over its shelf-life. Non-limiting examples for use herein include diazolidinyl urea, methylparaben, propylparaben, butylparaben, isobutylparaben, tetrasodium EDTA, and ethylparaben. The preservative may include a combination of parabens, such as methylparaben and propylparaben. In certain specific embodiments, the preservative is merfen and thiomersal; stabilized chlorine dioxide; and quaternary ammonium compounds such as benzalkonium chloride, cetyltrimethylammonium bromide and cetylpyridinium chloride, sorbic acid, paraben, phenoxyethanol, caprylyl glycol, ethylhexylglycerin, hexylene glycol or a combination thereof. In certain embodiments, the preservative is selected from sorbic acid, paraben, phenoxyethanol, caprylyl glycol, ethylhexylglycerin, and hexylene glycol, or a combination thereof. Examples of concentration ranges that the preservatives may be provided in include about 0.1% to about 10%, or about 0.5% to about 5% (w/v).

In some embodiments, the topical formulations are in the form of a suspension containing one or more polymers as suspending agents. Example polymers include water-soluble polymers such as cellulosic polymers, e.g., hydroxypropyl methylcellulose, and water-insoluble polymers such as cross-linked carboxyl-containing polymers. Certain formulations described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, the topical formulations include solubilizing agents to aid in the solubility of the cannabinoids and/or the one or more active sunscreen ingredients. The term “solubilizing agent” generally includes agents that result in formation of a micellar solution or a true solution of the agent. Certain acceptable nonionic surfactants, for example polysorbate 80, are useful as solubilizing agents. Examples include glycols, polyglycols, e.g., polyethylene glycol 400, and glycol ethers.

A topical formulation of a cannabinoid and one or more active sunscreen ingredients may also contain a gelling agent that increases the viscosity of the final solution. The gelling agent can also act as an emulsifying agent. The formulations can form clear gels and soft gels, which upon application to the skin can break down and deteriorate, affording gels that do not dry on the skin. Typically, the concentration and combination of gelling agents will depend on the physical stability of the finished product. Preferred concentration range of a gelling agent can be from about 0.01% to about 20%, more preferably from about 0.1% to about 10%, more specifically from about 0.5% to about 5% of the formulation (w/v). Non-limiting examples for use herein include classes of celluloses, acrylate polymers and acrylate crosspolymers. Preferably, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020).

In certain embodiments, the topical formulation contains one or more anti-oxidants, thiol containing compounds radical scavengers, and/or stabilizing agents, preferred concentration range from about 0.001% to about 0.1%, more preferably from about 0.1% to about 5% of the formulation (w/v). Non-limiting examples for use herein include butylatedhydroxytoluene, butylatedhydroxyanisole, ascorbyl palmitate, citric acid, vitamin E, vitamin E acetate, vitamin E-TPGS, ascorbic acid, sodium metabisulfite, tocophersolan and propyl gallate. More specifically the anti-oxidant can be ascorbyl palmitate, vitamin E acetate, vitamin E-TPGS, vitamin E or butylatedhydroxytoluene.

The topical formulation can be provided in any cosmetically suitable form, preferably as a gel, a lotion, or a cream, but also in an ointment or oil base, as well as a sprayable liquid form. In certain embodiments, the topical formulation be formulated as a balm (e.g., lip balm), a lotion, a liquid, a liquid spray (e.g., an aerosol spray), or a gel. In some particular embodiments, the topically administered formulation is formulated as a lotion. In other particular embodiments, the topically administered formulation is formulated as a lip balm.

In addition, the topical formulation can include any combination of compatible dermatologically acceptable additives commonly used, such as humectants, colorants, and the like, as well as botanicals, such as chamomile.

Humectants may be used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of hunectants can be from about 0.1% to about 70%, more preferably from about 5.0% to about 30%, more specifically from about 10% to about 25% of the formulation. Non-limiting examples for use herein include glycerin, polyhydric alcohols, hyaluronic acid, and silicone oils. In certain particular embodiments, the humectant may include glycerin, propylene glycol, glycereth-7 (a polyethylene glycol ether of glycerin), butylene glycol, sorbitol, maltitol, urea, flaxseed, algae extract, Aloe vera leaf extract, banana fruit extract, or any combination thereof.

In certain embodiments, the topical formulation has a pH of about 4 to about 7.5. In certain embodiments, the topical formulation has a pH of about 5 to about 5.5, about 5.5 to about 6, about 6 to about 6.5, or about 6.5 to about 7. Preferably, the topical formulation has a pH in the range of about 5 to about 7.

In some embodiments, the topical formulations include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the topical formulation in an acceptable range.

In some embodiments, the topical formulations include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the topical formulations include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Topical formulations including the cannabinoid and the one or more active sunscreen ingredients described herein may be manufactured by means of conventional mixing, dissolving, emulsifying, encapsulating, entrapping or lyophilizing processes. Formulations may be formulated in conventional manner using one or more physiologically acceptable carriers, diluents, excipients or auxiliaries which facilitate processing of ingredients into preparations that can be used dermatologically.

The protective effects of sunscreen formulations can be measured by the sun protection factor (SPF) rating of a given sunscreen formulation SPF is a relative value applied to sunscreens, indicating how much longer an individual can be exposed to sunlight without getting sunburned when wearing the sunscreen as compared to not wearing the sunscreen. For example, an SPF of 15 indicates that an individual who normally sunburns within 10 minutes should be able to remain in the sun for 115 minutes without becoming sunburned when they apply the sunscreen. SPF values are general estimates and actual results of skin protection vary depending on, for example, the time of the day and the amount of cloud cover. In some embodiments, the topical formulation has an SPF from about 5 to about 100, or from about 10 to about 55. In some embodiments, the topical formulation has an SPF of about 15, about 10, about 25, about 30, about 35, about 40, about 45, about 50, or about 55.

The protective effects of sunscreen formulations can be measured by other appropriate methods (see e.g., Augustin et al., Photochem Photobiol 66:853-9, 1997; Cole, Photodermatol Photoimmunol Photomed 17:2-10, 2001; and Schuch et al., Environmental Science & Technology 48: 11584-90, 2014).

### Packaged Sunscreens

Provided herein are packaged forms of the topical formulations described herein. The topical formulations may be packaged in containers (e.g., tubes and bottles) composed of hemp-based polymer material. Hemp-based polymers are renewable materials used in packaging as an alternative to petroleum-based packaging. In some embodiments, the container comprises material that is at least 25% hemp derived.

In some embodiments, the hemp-based polymer comprises hemp propylene, hemp ethylene, hemp acrylonitrile butadiene styrene, or a combination thereof. The hemp propylene may be a 25% hemp filled high-density polypropylene. The hemp ethylene may be a 25% hemp filled high-density polyethylene. The hemp acrylonitrile butadiene styrene may be a 25% hemp filled acrylonitrile butadiene styrene.

### Methods of Use

Provided herein are methods of using topical formulations comprising a cannabinoid and one or more active sunscreen agents as described above.

“Mammal” includes humans and both domestic animals such as laboratory animals and household pets, (e.g. cats, dogs, swine, cattle, sheep, goats, horses, rabbits), and non-domestic animals such as wildlife and the like. In certain specific embodiments, the mammal is a human. In certain specific embodiments, the mammal is a pet, such as a dog or cat.

A “subject” according to any of the above embodiments is a mammal. Mammals include but are not limited to, domesticated animals (e.g., cows, sheep, cats, dogs, and horses), primates (e.g., human and non-human primates such as monkeys), rabbits, and rodents (e.g., mice and rats). Preferably, the subject is a human.

In certain embodiments, the methods include protecting the skin of a subject from sunburn, comprising administering to the subject an effective amount of a topical composition as described herein.

In certain embodiments, the methods include protecting skin of a subject from ultraviolet (UV) radiation, comprising administering to the subject an effective amount of a topical composition as described herein. While not wishing to be bound by theory, the protective effects from sunlight of the topical formulations described herein include anti-inflammatory effects, and effects on epidermal differentiation and proliferation by the cannabinoid provided in the formulations.

In certain embodiments, administering the topical formulation results in a reduced likelihood of the skin undergoing carcinogenesis. While not wishing to be bound by theory, protective effects from sunlight of the topical formulations described herein include anti-tumorigenic effects of the cannabinoid provided in the formulations.

In particular embodiments, administering the topical formulation results in a reduced likelihood of a melanocyte in the skin undergoing carcinogenesis Malignant transformation of melanocytes (i.e., differentiated melanocytes or melanocyte progenitors) leads to melanoma.

In particular embodiments, administering the topical formulation results in a reduced likelihood of a keratinocyte in the skin undergoing carcinogenesis. Malignant transformation of keratinocytes may lead to squamous cell carcinoma or basal cell carcinoma.

The cannabinoid and one or more active sunscreen ingredients may be applied in a manner to achieve the intended protective effects from UV radiation. In some embodiments, the topical formulation is administered to skin of a subject prior to exposure of the skin to UV radiation. The topical formulations may be re-applied periodically (e.g., once an hour or once every two hours) during the time the subject is exposed to the UV.

In some embodiments, the topical formulations protect the skin from UVa radiation. In some embodiments, the topical formulations protect the skin from UVb radiation. In some embodiments, the topical formulations protect the skin from UVa and UVb radiation. Sunscreens that protect the skin from both UVa and UVb are known as broad spectrum sunscreens.

### EXAMPLES

### Example 1

### Protection from Uv-Induced Cellular Damage

The aim of this study is to test the effects of CBD on survival of keratinocytes and melanocytes following UV radiation.

Human primary epidermal melanocytes (ATCC PCS200-013) and keratinocytes (ATCC PCS 200-011) cell-lines were cultured using serum free complete growth media (ATCC PCS 200-030). Cells were cultured at 37 C.° with 5% CO₂. Cells were grown to 80% confluence before being washed with PBS buffer (37° C.) and trypsinized with EDTA-trypsin. A small aliquot of cells was stained with Trypan blue and counted using a haemocytometer. The remaining cells were plated in a 24 well plate with 300 uL of complete growth media (ATCC) for UV experimentation. Approximately 27*10\hat{ }3 cells were added to each well. Twenty-four hours after inoculation, individual wells were treated with one of three different concentrations of CBD isolate (CBD Brands, Jupiter, Fla.) or left untreated (control). The concentrations of CBD used were 1 μM, 4 μM and 8 μM, respectively.

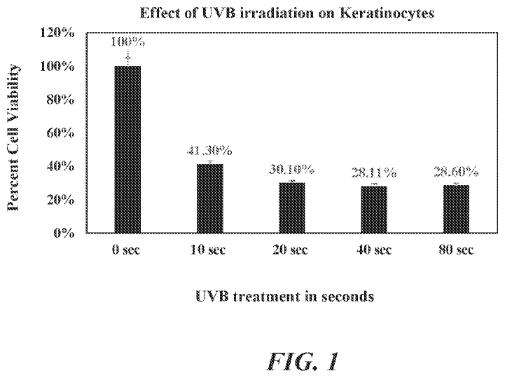

After CBD treatment for 1 hour, all cells were washed and with chilled (4° C.) PBS buffer. UV exposure experiments were conducted in chilled PBS buffer to avoid heat stress. Cells were irradiated with UVB light (Dermalight 80, UVB lamp). The distance of the lamp to the wells of the plate was kept constant at 3 cm. Exposure times were determined prior to the experiment, empirically, by exposing different wells to increasing UV exposure time and next conducting an MTT assay. FIG. 1 shows the keratinocyte cell viability at increasing UVB exposure times. The chosen exposure time was based on the exposure time that led to greater than 40% cell viability versus untreated cells determined by MTT assay. CBD treated cells were exposed to UV light for this predetermined time of 10 seconds (approximately 60 mJ/cm²). After exposure the effect of CBD on cell viability was evaluated by an MTT assay. Control cells were cultured in parallel with no CBD treatment, but underwent UVB irradiation.

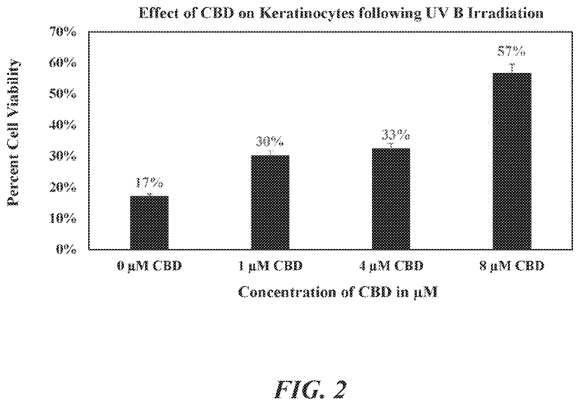

Treatment of primary keratinocytes with three different concentrations of CBD (1 μM, 4 μM and 8 μM, respectively) resulted in a dose dependent increase in cell viability after irradiation with UVB. Viability of cells not treated with CBD was the lowest at 17% determined from the MTT assay. The dose dependent protection provided by CBD in primary keratinocyte is depicted in FIG. 2.

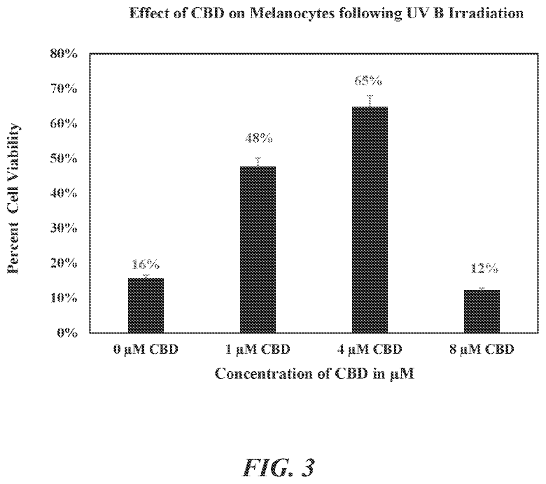

A similar UV protective effect was demonstrated for CBD in primary melanocytes below the highest concentration tested. Melanocytes treated with 0, 1, and 4 μM CBD demonstrated 16%, 48% and 65% viability, respectively, after UV irradiation. However Melanocytes treated with 8 μM CBD showed only 12% viability. A graphical representation of the cell viability calculated from MTT assay data is shown FIG. 3.

These results indicate a protective effect of CBD on the survival of keratinocytes and melanocytes following UV irradiation.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/005,854, filed on Apr. 6, 2020, and U.S. Patent Application No. 63/034,305, filed on Jun. 3, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A topical formulation, comprising a cannabinoid at an amount from about 0.1 mg/ml to about 50 mg/ml and one or more active sunscreen ingredients, wherein the one or more active sunscreen ingredients do not include oxybenzone.

2. The topical formulation according to claim 1, wherein the cannabinoid comprises a phytocannabinoid, an endocannabinoid, or a non-naturally occurring cannabinoid.

3. The topical formulation according to claim 1, wherein the cannabinoid is a phytocannabinoid, for example a cannabis-derived phytocannabinoid, for example one or more of Δ9-Tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), cannabiolsoin (CBE), and cannabicitran (CBT).

4-5. (canceled)

6. The topical formulation according to claim 3, wherein the cannabis-derived phytocannabinoid comprises CBD.

7. The topical formulation according to claim 3, wherein the cannabis-derived phytocannabinoid comprises a cannabinoid isolate having a total cannabinoid content of at least 95% cannabinoid (w/v).

8. The topical formulation according to claim 3, wherein the cannabis-derived phytocannabinoid comprises a cannabinoid isolate having a THC content of less than 0.3%.

9. The topical formulation according to claim 1, wherein the cannabinoid comprises an endocannabinoid, for example wherein the endocannabinoid comprises anandamide.

10. (canceled)

11. The topical formulation according to claim 1, wherein the cannabinoid comprises a non-naturally occurring cannabinoid, for example wherein the non-naturally occurring cannabinoid comprises CP55,940, WIN 55, 212-2, or nabilone.

12. (canceled)

13. The topical formulation according to claim 1, wherein the cannabinoid is at a concentration of about 1 mg/ml to about 10 mg/ml.

14. The topical formulation according to claim 1, wherein the one or more active sunscreen ingredients comprise at least one chemical sunscreen, optionally wherein the at least one chemical sunscreen is selected from avobenzone, homosalate, octisalate, octocrylene, or a combination thereof.

15. (canceled)

16. The topical formulation according to claim 14, wherein the at least one chemical sunscreen comprises:
avobenzone optionally at a concentration of about 0.5% to about 10% weight by volume (w/v); or
homosalate optionally at a concentration of about 5% to about 25% weight by volume (w/v); or
octisalate optionally at a concentration of about 0.5% to about 10% weight by volume (w/v); or
octocrylene optionally at a concentration of about 2% to about 20% weight by volume (w/v).

17. The topical formulation according to claim 1, wherein the one or more active sunscreen ingredients does not comprise avobenzone.

18-23. (canceled)

24. The topical formulation according to claim 1, wherein the one or more active sunscreen ingredients comprise at least one mineral sunscreen.

25. The topical formulation according to claim 24, wherein the at least one mineral sunscreen comprises one or both of zinc oxide, optionally at a concentration of about 5% to about 15% weight by volume (w/v), and titanium dioxide, optionally at a concentration of about 5% to about 15% weight by volume (w/v).

26-29. (canceled)

30. The topical formulation according to claim 1, wherein the one or more active sunscreen ingredients comprise homosalate, octisalate, octocrylene, and zinc oxide.

31. The topical formulation according to claim 30, wherein the one or more active sunscreen ingredients consist of homosalate, octisalate, octocrylene, and zinc oxide.

32. The topical formulation according to claim 1, wherein the one or more active sunscreen ingredients comprise avobenzone, homosalate, octisalate, and octocrylene.

33. The topical formulation according to claim 32, wherein the one or more active sunscreen ingredients consist of avobenzone, homosalate, octisalate, and octocrylene.

34. The topical formulation according to claim 1, wherein the topical formulation has a sun protection factor from about 5 to about 100, or from about 10 to about 55.

35. The topical formulation according to claim 1, wherein the topical formulation further comprises an emulsifier, a waterproofing agent, an emollient, a solvent, a perfume, an antioxidant, a preservative, or a combination thereof;
wherein:
the emulsifier optionally comprises sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polyglyceryl-4 oleate/PEG-8 propylene glycol cocoate, oleamide DEA, sodium glyceryl oleate phosphate, hydrogenated vegetable glycerides phosphate PEG-20 stearate, PEG-30 dipolyhydroxystearate, PEG-40 hydrogenated castor oil, PEG-60 hydrogenated castor oil, PEG-40 stearate, PEG-100 stearate, polyglyceryl-3 methylglucose distearate, polysorbate 20, polysorbate 60, polysorbate 80, potassium cetyl phosphate, DEA cetyl phosphate, ethomeen, dimethicone PEG-7 phosphate, or a combination thereof;
the emollient optionally comprises Simmondsia chinensis seed oil, glycerine, sorbitol, propylene glycol, isopropyl myristate, isopropyl palmitate, butylene glycol, caprys caprylic triglyceride, coco-caprylate/caprate, cocoglycerides, mineral oil, lanolin oil, coconut oil, cocoa butter, olive oil, aloe extract, banana fruit extract, jojoba oil, castor oil, a fatty acid such as oleic or stearic, fatty alcohol such as cetyl and hexadecyl, behenyl alcohol, diisopropyl adipate, hydroxybenzoate ester, benzoic acid ester, a C10-C16 alcohol, isononyl iso-nonanoate, a silicone such as dimethyl polysiloxane, caprylyl methicone, an ether such as polyoxypropylene butyl ethers or polyoxypropylene cetyl ether, C12-C15 alkyl benzoates, 1,2-hexanediol, acetyl glucosamine, or a combination thereof; and
the preservative optionally comprises sorbic acid, paraben, phenoxyethanol, caprylyl glycol, ethylhexylglycerin, and hexylene glycol, or a combination thereof.

36-41. (canceled)

42. The topical formulation according to claim 1, wherein the topical formulation has a pH of about 5 to about 7.

43. The topical formulation according to claim 1, wherein the topical formulation is in the form of a balm, a lotion, a liquid, or a gel.

44. The topical formulation according to claim 1, further comprising N-L-alpha-aspartyl-L-phenylalanine 1-methyl ester (APM) or a lower alkyl derivative thereof.

45. The topical formulation according to claim 44, wherein the APM or lower alkyl derivative thereof is at a concentration of about 0.05% to about 5% (w/v), preferably about 0.2 to about 2% (w/v).

46. The topical formulation according to claim 44, wherein a ratio of the APM or the lower alkyl derivative thereof to the cannabinoid in the formulation is in the range of about 4:1 to about 10:1 (by weight).

47. A packaged formulation comprising the topical formulation of claim 1, contained in a biodegradable package; optionally wherein the biodegradeable package comprises a hemp-based polymer, optionally wherein the hemp-based polymer comprises hemp propylene, hemp ethylene, acrylonitrile butadiene styrene, or a combination thereof.

48-49. (canceled)

50. A method of:
protecting the skin of a subject from sunburn, or
protecting skin of a subject from ultraviolet (UV) radiation, comprising administering to the subject an effective amount of a topical formulation of any of claim 1.

51. (canceled)

52. The method according to claim 50, wherein the administering is prior to exposure of the skin to UV radiation.

53. The method according to claim 50, wherein the administering reduces the likelihood of one or more of:
the skin undergoing carcinogenesis;
a melanocyte in the skin undergoing carcinogenesis:
a keratinocyte in the skin undergoing carcinogenesis.

54-55. (canceled)

56. The method according to claim 50, wherein the UV radiation comprises UVa, UVb, or both UVa and UVb.

57-58. (canceled)
